(19) United States
(12) Patent Application Publication
(10) Pub. No.: US 2020/0066386 A1
(43) Pub. Date: Feb. 27, 2020

(54) Interactive Medical Management System

(71) Applicant: MediMixer LLC, Agoura Hills, CA (US)

(72) Inventor: Keith Katz, Newbury Park, CA (US)

(21) Appl. No.: 16/545,098

(22) Filed: Aug. 20, 2019

Related U.S. Application Data
(60) Provisional application No. 62/721,758, filed on Aug. 23, 2018.

(51) Int. Cl. G16H 20/10; G08B 21/18

(52) U.S. Cl. CPC G16H 20/10; G08B 21/182

(57) ABSTRACT

A method for analyzing medical information includes receiving, via an interactive medical management (IMM) system, at least one medication event associated with a medication of a patient, and determining an amount of a supply of the medication possessed by the patient based on the medication event. The method also includes storing the medication event as part of a history of medication events, and producing, via the IMM system, an alert when the amount of the supply of the medication is less than a predetermined level.

### CROSS REFERENCE TO RELATED APPLICATIONS

This application claims priority to U.S. Provisional Patent Application No. 62/721,758, filed Aug. 23, 2018, which is incorporated herein by reference in its entirety.

### TECHNICAL FIELD

The present invention generally relates to systems and processes for managing, storing, and communicating medical information.

### BACKGROUND

Non-optimized prescription drug use and average pathway costs for a patient who experienced a treatment failure (TF), a new medical problem (NMP), or a TF and NMP, is extremely high and continues to increase. It is estimated that, in 2016, the annual cost of prescription drug-related morbidity and mortality resulting from non-optimized medication therapy was $528.4 billion in US dollars, with a plausible range of $495.3 billion to $672.7 billion. The average cost of an individual experiencing TF, NMP, or TF and NMP after initial prescription use were $2481 (range: $2233, $2742), $2610 (range: $2374, $2848) and $2572 (range: $2408, $2751), respectively. In addition to non-optimized prescription drug use, such costs can be further increased by an individual's failure to acquire or follow a personalized and monitored treatment plan.

Further, overall treatment plans can be inefficiently or incorrectly executed due to inherent limitations in various healthcare systems, such as the compartmentalized storage of information with various medical professionals, inefficiencies in communication between medical professionals, institutions, and patients, etc. Further, various aspects of a treatment plan can be hard to access for a patient on demand, which can result in a patient making incorrect or inadvisable choices that have negative long-term consequences, or consequences that are never communicated to medical professionals.

As a result, there is a need for a process and system for receiving, storing, and communicating medical information to the user and relevant medical professionals and organizations.

### SUMMARY

An embodiment of the present disclosure is a method for analyzing medical information. The method includes receiving, via an interactive medical management (IMM) system, at least one medication event associated with a medication of a patient, and determining an amount of a supply of the medication possessed by the patient based on the medication event. The method also includes storing the medication event as part of a history of medication events, and producing, via the IMM system, an alert when the amount of the supply of the medication is less than a predetermined level.

A further embodiment of the present disclosure is method for analyzing medical information. The method includes receiving, via an interactive medical management (IMM) system, a medical parameter of a patient from a remote medical device, storing the medical parameter as part of a medical profile of the patient, and sending the medical profile to a computing device located at a healthcare provider premises.

Another embodiment of the present disclosure is an interactive medical management (IMM) system. The system includes a processor, at least one human-machine interface (HMI) device, and at least one memory storing instructions that, when executed, cause the processor to perform a process. The process includes receiving at least one medication event associated with a medication of a patient, and determining an amount of a supply of the medication possessed by the patient based on the medication event. The method also includes storing the medication event as part of a history of medication events, and producing an alert when the amount of the supply of the medication is less than a predetermined level.

### BRIEF DESCRIPTION OF THE DRAWINGS

The foregoing summary, as well as the following detailed description of illustrative embodiments of the systems and methods of the present disclosure, are better understood when read in conjunction with the appended drawings. It should be understood, however, that the present disclosure is not limited to the precise schematics and arrangements shown. In the drawings:

FIG. 1 is a block diagram of an interactive medical management system according to an embodiment of the present disclosure;

FIG. 2 is a schematic illustration of an example hardware and network configuration in which the interactive medical management system shown in FIG. 1 can be implemented for managing and communicating medical information;

FIG. 3 is a flow chart illustrating a first exemplary process that may be performed by the interactive medical management system of FIG. 1;

FIG. 4 is a flow chart illustrating a continuation of the exemplary process shown in FIG. 3;

FIG. 5 is a flow chart illustrating a second exemplary process that may be performed by the interactive medical management system of FIG. 1; and

FIG. 6 is a flow chart illustrating a third exemplary process that may be performed by the interactive medical management system of FIG. 1.

While the flow and sequence diagrams presented herein show an organization designed to make them more comprehensible by a human reader, those skilled in the art will appreciate that actual data structures used to store this information can differ from what is shown, in that they, for example, can be organized in a different manner; can contain more or less information than shown; can be compressed and/or encrypted; etc.

Some components and/or operations can be separated into different blocks or combined into a single block for the purposes of discussion of some of the embodiments. Moreover, while the various embodiments are amenable to various modifications and alternative forms, specific embodiments have been shown by way of example in the drawings and are described in detail below. The intention, however, is not to limit the particular embodiments described. On the contrary, the embodiments are intended to cover all modifications, equivalents, and alternatives falling within the scope of the disclosed embodiments as defined by the appended claims.

### DETAILED DESCRIPTION OF ILLUSTRATIVE EMBODIMENTS

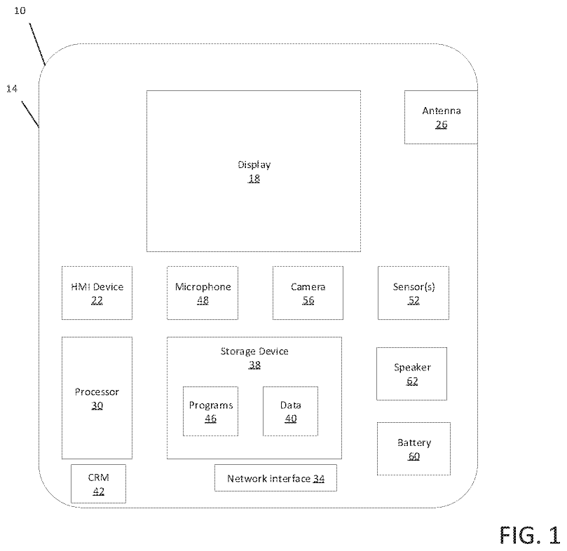

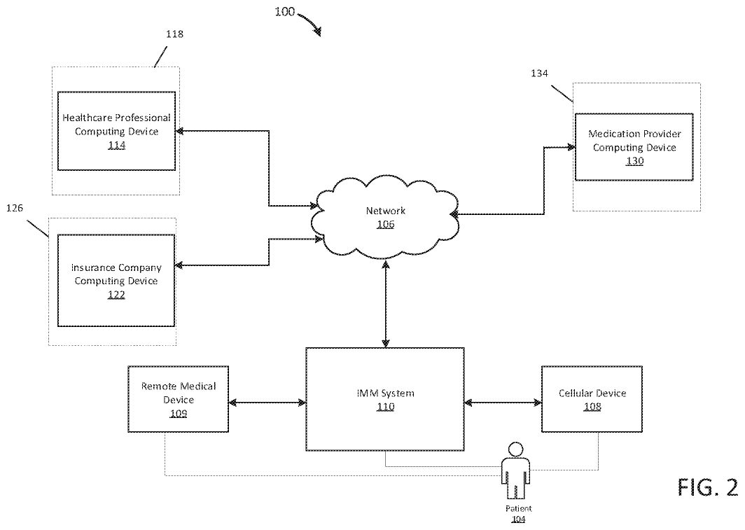

FIG. 1 shows a diagram of an exemplary interactive medical management (IMM) system 10, while FIG. 2 shows an example hardware and network configuration in which the IMM system 10 can be implemented. As shown, the IMM system 10 can include a housing 14 configured to retain various components of the IMM system 10 and to which various components of the IMM system 10 can be attached. The housing 14 can have the shape of a hollow cube with rounded and/or curved edges, though other shapes for the housing 14 are contemplated. The housing 14 can be comprised of thing plastic, metal, etc., though other materials can be utilized as desired. The housing 14 can be sized such that the IMM system 10 is easily transportable by a patient and can be disposed upon a countertop, table, etc. The IMM system 10 can include a display 18, HMI device 22, antenna 26, processor 30, network interface 34, storage device 38, computer readable medium 42, microphone 48, sensor 52, camera 56, battery 60, and speaker 62.

The IMM system 10 can include a display 18 attached to the housing 14 such that the display 18 is visible by a patient using the IMM system 10. The display 18 can include one or more devices for displaying information, including but not limited to, liquid crystal displays (“LCD”), light emitting diode (“LED”) screens, organic light emitting diode (“OLED”) screens, and other known display devices. The display 18 can further include touch-screen capabilities, such that the display 18 can receive patient input. The display 18 can be configured to display various types of medical information, such as information related to medical referrals, including medical professionals and their associated contact information, ratings of medical professionals, insurance information, etc. The display 18 can also be configured to provide alerts, which can be reminders and/or notifications for appointments, etc. The display 18 can also display information related to medication, such as prescription information, dosage information, side effects, next schedule dosage time, dosage frequency, last time a dosage was taken, etc.

The IMM system 10 can further include human-machine interface (HMI) devices 22 can include one or more devices that allow the IMM system 10 to send and receive information. The HMI devices 22 can include, for example, a pointing device, keyboard, buttons, switches, and/or a touchscreen panel. HMI devices 22 can also include one or more communication modules (not shown) for sending and receiving information via antenna 26 from other devices by, for example, establishing wired or wireless connectivity between IMM system 10 to network 106, or by establishing direct wired and/or wireless connections between the IMM system 10 and one or more remote medical device 109. Direct connections can include, for example, Bluetooth™, Bluetooth LE™, WIFI, near field communications (NFC), or other known communication process which provide a medium for transmitting data between separate devices.

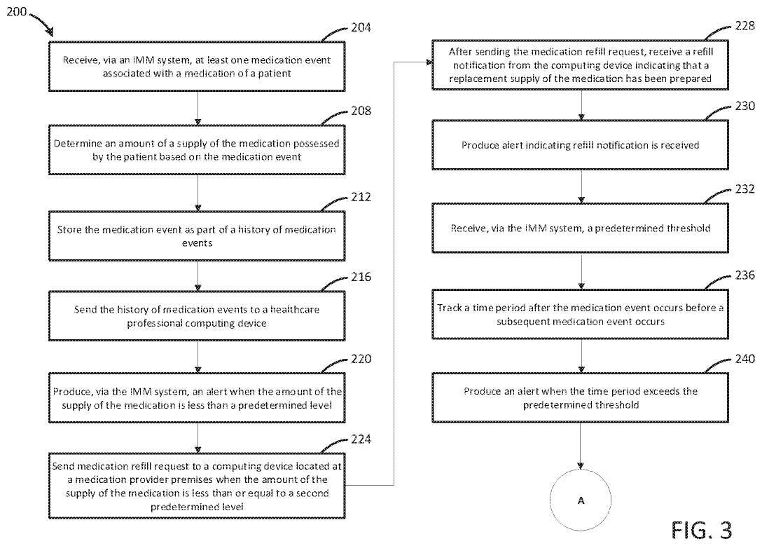

The processor 30 can be one or more known processing devices, such as microprocessors manufactured by Intel™ or AMD™ or licensed by ARM. Processor 30 can constitute a single core or multiple core processors that executes parallel processes simultaneously. For example, processor 30 can be a single core processor configured with virtual processing technologies. In certain embodiments, processor 30 can use logical processors to simultaneously execute and control multiple processes. Processor 30 can implement virtual machine technologies, or other known technologies to provide the ability to execute, control, run, manipulate, store, etc. multiple software processes, applications, programs, etc. One of ordinary skill in the art would understand that other types of processor arrangements could be implemented that provide for the capabilities disclosed herein. The operation of the processor 30 will be better understood with reference to the processes 200, 300, 400 described in relation to FIGS. 3-6 below.

In some embodiments, processor 30 may be configured to receive data (e.g., medical data) over network 106 via network interface 34. Processor 30 can also be configured to store the data in storage device 38, which may be accessed by over network 106 for further processing. Storage device 38 can be any combination of databases, datasets, flat files, etc. stored in computer memories (e.g., RAM. ROM, or Flash memory), hard drives, remote network servers, or other data storage components or computer-readable storage devices. Although a single storage device 38 is illustrated in FIG. 1, it will be appreciated that the storage area may include multiple storage devices 38. Storage device 38 may store data 40 that is utilized, generated, or modified by the IMM system 10, such as various types of medical data as will be described further below. The IMM system 10 can include a computer readable medium (CRM) 42. CRM 42 may include computer-readable instructions encoded for performing an analysis of data 40. CRM 42 may be coupled to processor 30, which executes the instructions and programs 46 stored on storage device 38 in order to implement the functionality disclosed herein.

In some embodiments, IMM system 10 can include a microphone 48 and one or more sensors 52 for collecting environmental data. For example, microphone 48 can be configured to capture audio from the environment proximate to IMM system 10, such as instructions, data, or queries from the patient. IMM system 10 can be configured to process the captured audio, as will be described below. Sensor(s) 52 can include other environmental sensors such as, for example, GPS receivers, location-based data receivers, accelerometers, and gyroscopes. For example, sensor(s) 52 can be configured to detect positioning data indicative of a location of a remote medical device 109, which will be described below. The IMM system 10 can further include a camera 56 configured to allow a user, in conjunction with the microphone 48 and display 18, engage in two-way communication with a healthcare professional, as will be described below. The camera 56 can also be utilized for security purposes, such as to allow for unlocking and/or powering on the IMM system 10 based upon retina and/or facial recognition. The camera 56 can further be utilized to receive various types of input from the user, such as lip reading, sign language recognition, hand signal recognition, etc. The IMM system 10 can further include a speaker 62 configured to produce audial signals to alert the patient of various conditions, as will be described further below. In some embodiments, processor 30 can use data collected by microphone 48, sensors 52, camera 56, and/or display 18 to control or modify functions of program(s) 46.

The IMM system 10 can be powered via a wired connection (not shown) to a power source, which can be permanently affixed or detachable from the IMM system 10. Alternatively or additionally, the IMM system 10 can include a battery 60, which can be rechargeable or disposable, for powering the IMM system 10 permanently or during periods during which a wired power connection is unavailable, such as during power outages.

Referring to FIG. 2, a schematic illustration of an exemplary environment including the IMM system 10 is depicted. As shown, the IMM system 10 can be in wired and/or wireless connection with a patient or caregiver cellular device 108 to transmit instructions and/or information therebetween, as will be described further below. The cellular device 108 can run an application that sends and receives various types of medical information to the IMM system 10, based upon input received by the cellular device 108, or by processes performed by the processor of the IMM system 10. For example, the application run by the cellular device 108 can be configured to send and/or receive information including notifications, dosage information, dosage frequency, medication type, messages for medical professionals, scheduling information, etc. The cellular device 108 can also be utilized to control any of the below-described processes of the IMM system 10.

Further, direct wired and/or wireless connections between the IMM system 10 and one or more remote medical device 109 can exist. The remote medical device 109 can include one or more of a thermometer, stethoscope, oxygen sensor, pulsometer, heart monitor, breathing monitor, blood pressure monitor, blood oxygen monitor, blood sugar monitor, deep breathing lung exerciser, pulse oximeter, and a breath measurement system. Though specific devices are listed, it is contemplated that the remote medical device 109 can include various other types of medical equipment.

Through the network 106, the IMM system 10, and likewise the remote medical device 109 and cellular device 108, can be in signal communication with various computing devices and systems located remotely from the IMM system 10. The network 106 may comprise one or more public networks (e.g., the Internet) and/or one or more private networks. A private network may include a wireless local area network (WLAN), a local area network (LAN), a wide area network (WAN), a cellular network, or an intranet. The network 106 may comprise wired network(s) and/or wireless network(s).

The IMM system 10 can be in signal communication with a healthcare professional computing device 114 through the network 106, where the healthcare professional computing device 114 can be owned and/or operated by a healthcare professional. The healthcare professional can be a doctor, nurse, dentist, surgeon, physical therapist, etc. In some instances, the healthcare professional can be an individual with prescription authority in the relevant jurisdiction. The healthcare professional computing device 114 can be located at a healthcare professional premises 118, which can be a doctor's office, hospital, dentist's office, urgent care facility, etc. The healthcare professional computing device 114 can be one or more computing devices, such as handheld computing devices, tablet computers, as well as one or more off-premises computing devices, such as one or more servers in communication with one or more on-premises computing devices.

The IMM system 10 can also be in signal communication with an insurance company computing device 122 through network 106, where the insurance company computing device 122 can be owned and/or operated by insurance personnel. The insurance personnel can be an insurance adjuster, insurance representative, etc. The insurance company computing device 122 can be located at an insurance company premises 126, which can be an office of the insurance company. The insurance company computing device 122 can be one or more computing devices, such as one or more handheld computing devices, tablet computers, as well as one or more off-premises computing devices, such as one or more servers in communication with one or more on-premises computing devices.

The IMM system 10 can further be in signal communication with a medication provider computing device 130 through network 106. The medication provider computing device 130 may be associated with respective medication providers. A medication provider may include a pharmacy, a pharmacy chain or brand, a brick-and-mortar pharmacy, a mail-order pharmacy, an online pharmacy, or a tele-pharmacy, and may be located at a medication provider premises 134. Some medication providers may exhibit several of these characteristics, such as a pharmacy company that operates a number of brick-and-mortar pharmacies and also provides mail-order pharmacy services. A medication provider computing device 130 may receive medication information and respond to the medication information with an offer to provide the medication at a specified price. A medication provider computing device 130 may receive an order and associated payment for a medication and fulfill such an order. A medication provider may comprise or be associated with a medication distribution system. A medication distribution system may package and deliver the medication to the patient, such as via mail to the patient's residence. A medication provider computing device 130 may comprise one or more computing devices, such as one or more networked servers.

Additionally, the cellular device 108 can be configured to allow for direct communication with the remote medical device 109, healthcare professional computing device 114, insurance company computing device 122, and/or medication provider computing device 130 through the IMM system 10 and/or network 106. Accordingly, a user can provide to or request information from the healthcare professional computing device 114, insurance company computing device 122, and/or medication provider computing device 130 through the application run by the cellular device 108.

FIG. 3 is a flow chart illustrating a first exemplary method 200 that may be performed by the processor 30 of the IMM system 10 of FIGS. 1 and 2. Method 300 may comprise a method for analyzing medical information using the IMM system 10, and in particular managing and analyzing information related to medication of a patient 104.

In step 204, information related to a medication event associated with a medication of the patient 104 is received. The medication event may comprise the fact that a dosage of the medication has been taken by the patient 104, prescription of the medication has been refilled, etc. The information can be directly input into the IMM system 10 by the patient 104, such as through audial transmission through the microphone 48, interaction with the HMI device 22, or interaction with the display 18 when the display 18 has touch screen capabilities. Alternatively or additionally, the patient 104 can provide the medication event to the cellular device 108, from which the IMM system 10 receives the medication event.

At step 208, the processor 30 can determine an amount of a supply of the medication possessed by the patient 104 based on the received medication event. The processor 30 can perform this determination based data 40 stored in the storage device 38, which can include the instructed dosage of medication, amount of the medication possessed by the patient 104 when filled, past medication events, etc. When this determination is made, the IMM system 10 can provide an output through the display 18 or speaker 62 that includes information related to the supply of the medication, such as total amount of medication remaining, time until next prescription refill, etc. In step 212, the IMM system 10 can then store the medication event in the storage device 38 as part of a history of medication events provided to the IMM system 10 by the patient.

At step 216, the IMM system 10 can send the history of medication events to the healthcare professional computing device 114 that is located at the healthcare professional premises 118. This step can be performed either upon request by the healthcare professional, automatically through a request from healthcare professional computing device 114 or at a specified regular interval, or upon instructions received from the patient 104. At step 220, the IMM system 10 can produce an alert when the amount of the supply of the medication is less than a predetermined level. This alert can take the form of a visual alert produced by the display 18, an audial alert produced by the speaker 62, etc. The alert can also take the form of a notification pushed to the application run by the cellular device 108.

At step 224, the IMM system 10 can send a medication refill request to a medication provider computing device 130 located at a medication provider premises 134 when the amount of the supply of the medication is less than or equal to a second predetermined level. In some embodiments, the second predetermined level can be different than the first predetermined level, though it is contemplated that the first and second predetermined levels can be equal. This ensures that the patient 104 can have a constant supply of medication and the medication plan will not be interrupted. In alternative embodiments, the IMM system 10 can send a refill request to the medication provider computing device 130 upon instruction from the patient 104. At step 228, after sending the medication refill request, the IMM system 10 can receive a refill notification from the medication provider computing device 130 indicating that a replacement supply of the medication has been prepared. The notification can represent the ability to pick the medication up at the medication provider premises 134, shipping of the medication to the patient 104, etc. After step 228, at step 230 the IMM system 10 can produce an alert when the refill notification is received. This alert can be provided as an audial notification from the speaker 62, a visual notification on the display 18, etc. Alternatively or additionally, this alert can be provided as a notification from the application run by the cellular device 108.

The IMM system 10 can also ensure that a patient 104 does not miss schedule doses of a medication and thus does not deviate from a treatment plan developed by a medical professional. To do this, at step 232 the IMM system 10 can receive a predetermined threshold that represents the intended time period between doses of a medication, or alternatively scheduled times at which a dosage of medication is to be taken. The predetermined threshold can be received from the patient 104 through an input into the microphone 48, HMI device 22, or display 18 of the IMM system 10. Alternatively, the predetermined threshold can be received from the medication provider computing device 130 or the healthcare professional computing device 114. In step 236, the IMM system 10 can track a time period after the medication event occurs before a subsequent medication event occurs. When the subsequent medication event occurs within or according to the predetermined threshold, the IMM system 10 can produce a positive reinforcement signal using the display 18, speaker 62, etc. Alternatively, the IMM system 10 can initiate a reward, such as through crediting a rewards account, which can be through the associated insurance carrier, a third-party retailer, etc. However, then the time period exceeds a predetermined threshold, at step 240 the IMM system 10 can produce an alert. This alert can be provided as an audial notification from the speaker 62, a visual notification on the display 18, etc. Alternatively or additionally, this alert can be provided as a notification from the application run by the cellular device 108. The IMM system 10 can also send a notification of a deviation from the medication plan to the healthcare professional computing device 114, insurance company computing device 122, and/or medication provider computing device 130.

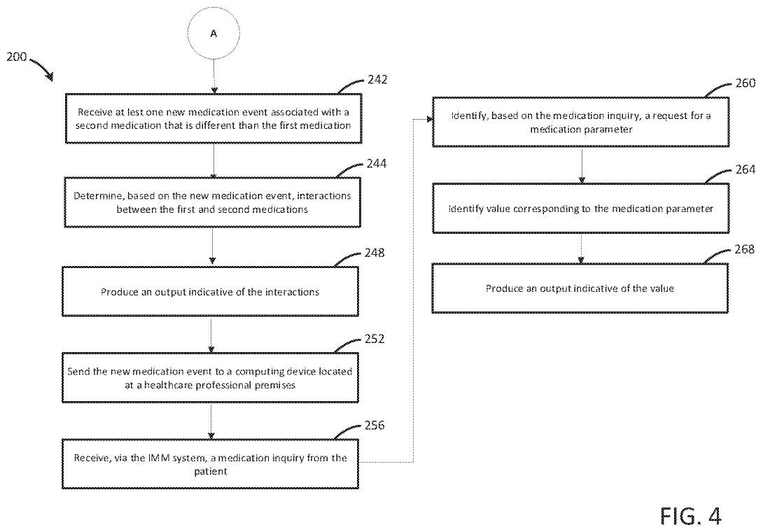

Process 200 is continued in FIG. 4. The IMM system 10 can further be configured to analyze interactions between various medications taken by the patient 104, convey these results to the patient 104, as well as notify healthcare professionals as to additional medications, whether over the counter or prescription. At step 242, the IMM system 10 can receive at least one new medication event associated with a second medication that is different than the first medication. At step 244, based on the new medication event, the IMM system 10 can determine interactions between the first and second medications. This determination can be based upon internally stored information within the storage device 38, web-based searches, or electronic communication with a healthcare professional through the network 106.

In step 248, the IMM system 10 can produce an output indicative of the interactions between the first and second medications. This output can be provided as an audial notification from the speaker 62, a visual notification on the display 18, etc. Alternatively or additionally, this output can be provided as a notification from the application run by the cellular device 108. As such, the patient 104 can be easily apprised of the interactions between various medications and make informed decisions as to whether to begin a new treatment regimen or whether potential adverse consequences will be encountered. At step 252, the IMM system 10 can send the new medication event to the healthcare professional computing device 114. Alternatively or additionally, the IMM system 10 can send the new medication event to the medication provider computing device 130. As a result, healthcare professionals that are responsible for developing comprehensive treatment plans for the patient 104 can be constantly apprised of new developments in a treatment regimen and can make appropriate changes or recommendations to an overall treatment plan accordingly.

The IMM system 10 can also define a comprehensive repository for medication information related to the patient 104, allowing the patient 104 and/or any healthcare professional instant access to a variety of information related to the treatment plan of the patient 104. Specifically, in step 256, the IMM system 10 can receive a medication inquiry from the patient 104. The medication inquiry can be received from the patient 104 through display 18, microphone 48, HMI device 22, etc. Alternatively, the medication inquiry can be received from the application run by the cellular device 108 as a result of user input. The medication inquiry can comprise a request for any variety of information, such as next scheduled dosage time, dosage amount, medication price, amount of medication remaining, etc.

At step 260, the IMM system 10 can identify, based on the medication inquiry, a request for a medication parameter. Then, in step 264, the IMM system 10 can identify a value corresponding to the medication parameter for the particular medication at issue. The value can be stored locally in the storage device 38 of the IMM system 10 or can be retrieved through the network 106. If ascertaining the value requires consultation with medical professionals, the IMM system 10 can send an appropriate inquiry to the healthcare professional computing device 114 and/or the medication provider computing device 130. After the medication parameter has been retrieved, at step 268 the IMM system 10 can produce an output indicative of the value. This output can be provided as an audial notification from the speaker 62, a visual notification on the display 18, etc. Alternatively or additionally, this output can be provided as a notification from the application run by the cellular device 108. As a result, the patient 104 can have all information related to his/her medication treatment plan easily accessible, which can lead to decreased missteps in a treatment plan or misuse of medication. In addition to the patient 104, a healthcare professional, via the healthcare professional computing device 114 and/or the medication provider computing device 130, or third party via the insurance company computing device 122 can retrieve information related to the medication plan of the patient 104 from the IMM system 10 through the network 106 so as to reliably maintain current records of the treatment plan of the patient 104.

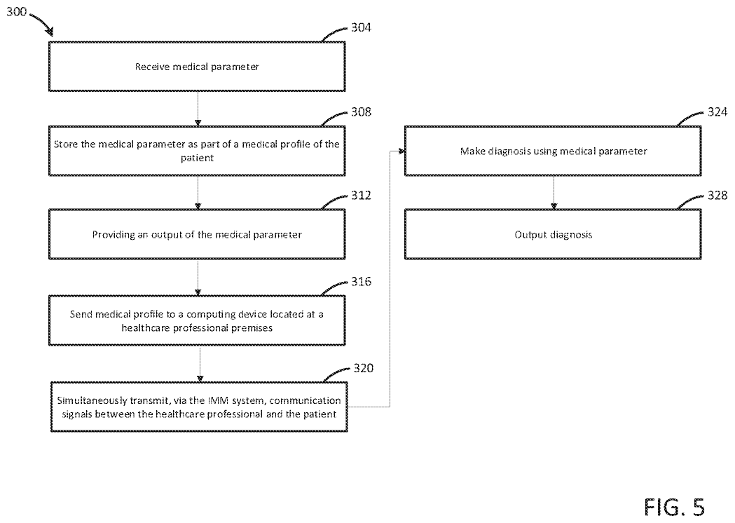

Now referring to FIG. 5, a flow chart is depicted illustrating a second exemplary process 300 that may be performed by the processor 30 of the IMM system 10 of FIGS. 1 and 2. Process 300 may comprise a method for receiving and sharing medical information of a patient 104 using the IMM system 10.

In step 304, the IMM system 10 receives a medical parameter. The medical parameter can comprise a wide variety of information, such as all varieties of medical records related to the patient 104, appointment times and history, insurance information, preferred medical professional information, etc. The medical parameter can be directly input by the patient 104 into the IMM system 10 via the HMI device 22, display 18, and/or microphone 48. Alternatively, the medical parameter can be input by the patient 104 or an associated caregiver into the application run by the cellular device 108. Further, the medical parameter can be received by the IMM system 10 from the healthcare professional computing device 114, insurance company computing device 122, and/or medication provider computing device 130. This can be done automatically, upon request by the patient 104, or upon request by a healthcare professional or insurance personnel.

Alternatively, step 304 can involve receiving the medical parameter from a remote medical device 109. As stated previously, the remote medical device 109 can include one or more of a thermometer, stethoscope, oxygen sensor, pulsometer, heart monitor, breathing monitor, blood pressure monitor, blood oxygen monitor, blood sugar monitor, deep breathing lung exerciser, pulse oximeter, and a breath measurement system. However, it is also contemplated that other patient monitoring devices not listed herein can be utilized as the remote medical device 109. As a result, the IMM system 10 can receive health parameters from the remote medical device 109 related to the condition of the patient, such as pulse, heart rate, blood sugar, temperature, blood oxygen level, etc. At step 308, the IMM system 10 can store the medical parameter as part of a medical profile of the patient 104. This can involve storing the medical parameter as a new value, an update to a previous vale, part of a value array, etc.

At step 312, the IMM system 10 can provide an output of a desired medical parameter stored in the storage device 38 upon request by the patient 104. The patient 104 can input such a request via the HMI device 22, display 18, and/or microphone 48. The output reflecting the medical parameter can be provided as an audial notification from the speaker 62, a visual notification on the display 18, etc. Alternatively or additionally, this output can be provided as a notification from the application run by the cellular device 108. Additionally, at step 316, the IMM system 10 can send the medical profile to a healthcare professional computing device 114 located at a healthcare professional premises 118. The medical profile communicated by the IMM system 10 can comprise the entirety of the medical profile stored in the storage device 38 or any portion thereof, as specified by the patient 104 and/or the healthcare professional. As such, the healthcare professional can monitor medical parameters related to the patient 104 so as to be constantly apprised of the status of a treatment process. This can allow the healthcare professional to recognize issues in a real-time basis, rather than solely at in-person appointments, to prevent undue harm and cost to the patient.

At step 320, the IMM system 10 can simultaneously transmit communication signals between the healthcare professional and the patient 104. These communication signals can comprise audial signals via the microphone 48 and speaker 62, visual signals via the display 18, or a combination thereof. As such, the patient 104 and healthcare professional can communicate in real time about the status of a treatment plan and/or medical parameter of the patient so as to identify and implement treatment options without the need for an in-person appointment, thereby saving both the patient 104 and the healthcare professional time and effort. During step 320, the healthcare professional can request the patient 104 to take additional medical parameters using any selection of the remote medical devices 109, where the medical parameters can be instantly uploaded to the IMM system 10 and sent to the healthcare professional computing device 114, thereby allowing the healthcare professional to engage in real-time diagnosis of the patient 104.

At step 324, the IMM system 10 can itself make the diagnosis using the medical parameter. The IMM system 10 can make this diagnosis using information stored in the storage device 38 including risk factors of the particular patient 104, medical history of the patient 104, database of medical conditions and symptoms, information received via the network 106, etc. The IMM system 10 can perform step 324 using artificial intelligence (AI). Additionally, at step 328, the IMM system 10 can output the diagnosis. The output reflecting the diagnosis can be provided as an audial notification from the speaker 62, a visual notification on the display 18, etc. Alternatively or additionally, this output can be provided as a notification from the application run by the cellular device 108.

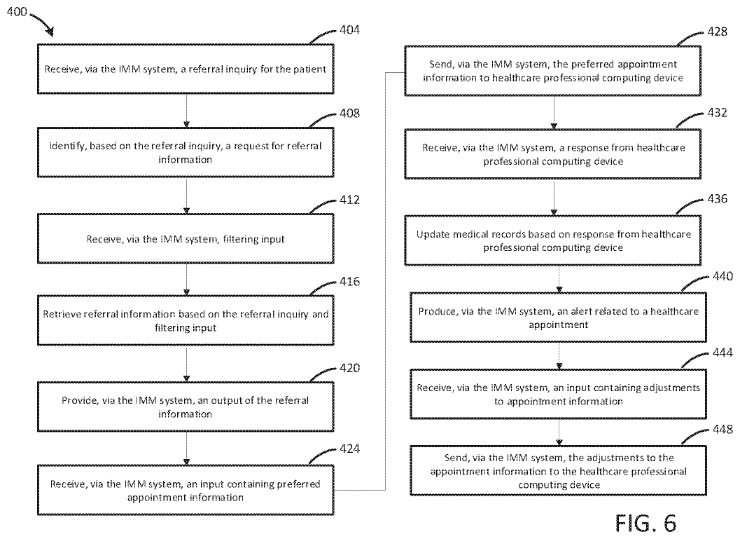

Now referring to FIG. 6, a flow chart is depicted illustrating a third exemplary method 400 that may be performed by the processor 30 of the IMM system 10 of FIGS. 1 and 2. Process 400 may comprise a method for receiving referrals and scheduling appointments for a patient 104 using the IMM system 10.

At step 404, the IMM system 10 can receive a referral inquiry for the patient 104. The referral inquiry can relate a need for patient 104 to schedule an appointment or have a consultation with a particular medical personnel. The referral inquiry can comprise a particular type of doctor needed, type of procedure needed, type of medical issue encountered, etc. The referral inquiry can be directly input by the patient 104 into the IMM system 10 via the HMI device 22, display 18, and/or microphone 48. Alternatively, the referral inquiry can be input by the patient 104 or an associated caregiver into the application run by the cellular device 108. Further, the referral inquiry can be received by the IMM system 10 from the healthcare professional computing device 114, insurance company computing device 122, and/or medication provider computing device 130. This can be done automatically, upon request by the patient 104, or upon request by a healthcare professional or insurance personnel.

At step 408, the IMM system 10 can identify, based on the referral inquiry, a request for referral information. At step 412, the IMM system 10 can receive filtering input. Filtering input can comprise a variety of information related to the preference of the patient 104 related to the referral inquiry such that the IMM system 10 produces the referral results most favorable to the patient 104. Such information can include insurance preferences (in vs. out of network), maximum distance from the residence of the patient to a healthcare provider premises, desired appointment time, minimum healthcare professional user rating, etc. Like the referral inquiry, the filtering input can be directly input by the patient 104 into the IMM system 10 via the HMI device 22, display 18, and/or microphone 48. Alternatively, the filtering input can be input by the patient 104 or an associated caregiver into the application run by the cellular device 108. Further, the filtering input can be received by the IMM system 10 from the healthcare professional computing device 114, insurance company computing device 122, and/or medication provider computing device 130. This can be done automatically, upon request by the patient 104, or upon request by a healthcare professional or insurance personnel.

At step 416, the IMM system 10 can retrieve referral information based on the referral inquiry and the filtering input. This referral information can comprise the healthcare providers that the processor 30 determines best matches the referral inquiry provided by the patient 104, as limited by the filtering input. The referral information can be stored locally on the storage device 38 of the IMM system 10. Alternatively or additionally, referral information can be received through the network 106 from the healthcare professional computing device 114 and/or the insurance company computing device 122. Once the referral information has been received, at step 420 the IMM system 10 can provide an output of the referral information. The output can include a name, location, hours, rating, insurance network status, and/or specialty of a healthcare provider or selection of healthcare providers, as well as other pertinent information. As a result, the IMM system 10 can comprise an encompassing healthcare solution that allows a patient 104 to easily and efficiently find and connect with healthcare providers that best match their particular healthcare needs and preferences. This output of referral information can be provided as an audial notification from the speaker 62, a visual notification on the display 18, etc. Alternatively or additionally, this output can be provided as a notification from the application run by the cellular device 108.

After the output of the referral information has been produced, at step 424 the IMM system 10 can receive an input containing preferred appointment information. This information can include the selected healthcare provider, desired appointment time and date, etc. The preferred appointment information can be directly input by the patient 104 into the IMM system 10 via the HMI device 22, display 18, and/or microphone 48. Alternatively, the filtering input can be input by the patient 104 or an associated caregiver into the application run by the cellular device 108. The IMM system 10 can then at step 428 send the preferred appointment information to the healthcare professional computing device 114. Though one healthcare professional computing device 114 is specifically described, it should be appreciated that the network 106 can connect the IMM system 10 to a plurality of healthcare professional computing devices 114. It should also be appreciated that step 428 involves sending the preferred appointment information to the healthcare professional computing device 114 corresponding to the healthcare professional selected by the patient 104 from the referral information.

Step 432 involves receiving, via the IMM system 10, a response from the healthcare professional computing device 114 in response to the preferred appointment information received from the IMM system 10. The response can include confirmation or denial of the preferred appointment information, alternative appointment information, or additional information provided by the healthcare professional. As such, the IMM system 10 provides a patient 104 with easy and automatic scheduling functionality that requires minimum effort, thus reducing hurdles to obtaining medical treatment for the patient 104, saving the patient 104 and insurance companies both time and money. At step 436, the IMM system 10 can update medical records stored in the storage device 38 based on the response received from the healthcare professional computing device 114. For example, the IMM system 10 can update a stored calendar of the patient 104 to reflect a new, changed, or canceled appointment.

The IMM system 10, in step 440, can also be configured to produce an alert related to a healthcare appointment. As such, the patient 104 can be reminded by the IMM system 10 of upcoming appointments so that appointments are not missed. This alert can take the form of a visual alert produced by the display 18, an audial alert produced by the speaker 62, etc. The alert can also take the form of a notification pushed to the application run by the cellular device 108.

The IMM system 10 can also possess the functionality of allowing the patient 104 to communicate with medical professionals so as to make changes to or cancel existing appointments. At step 444, the IMM system 10 can receive an input containing adjustments to appointment information. The information can be directly input into the IMM system 10 by the patient 104, such as through audial transmission through the microphone 48, interaction with the HMI device 22, or interaction with the display 18 when the display 18 has touch screen capabilities. Alternatively or additionally, the patient 104 can provide the medication event to the cellular device 108, from which the IMM system 10 receives the medication event. At step 448, the IMM system 10 sends the adjustments to the appointment information to the healthcare professional computing device 114. The adjustments can include time changes, day changes, cancellations, etc. This schedule integration allows the patient 104 to easily and efficiently make changes to existing appointments.

One skilled in the art will appreciate that the systems and methods disclosed herein may be implemented via a computing device that may comprise, but are not limited to, one or more processors, a system memory, and a system bus that couples various system components including the processor to the system memory. In the case of multiple processors, the system may utilize parallel computing.

For purposes of illustration, application programs and other executable program components such as the operating system are illustrated herein as discrete blocks, although it is recognized that such programs and components reside at various times in different storage components of the computing device, and are executed by the data processor(s) of the computer. An implementation of service software may be stored on or transmitted across some form of computer readable media. Any of the disclosed methods may be performed by computer readable instructions embodied on computer readable media. Computer readable media may be any available media that may be accessed by a computer. By way of example and not meant to be limiting, computer readable media may comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by a computer. Application programs and the like and/or storage media may be implemented, at least in part, at a remote system.

As used in the specification and the appended claims, the singular forms “a,” “an” and “the” include plural referents unless the context clearly dictates otherwise. Ranges may be expressed herein as from “about” one particular value, and/or to “about” another particular value. Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect.

It will be apparent to those skilled in the art that various modifications and variations may be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed:

1. A method for analyzing medical information, the method comprising:
receiving, via an interactive medical management (IMM) system, at least one medication event associated with a medication of a patient;
determining an amount of a supply of the medication possessed by the patient based on the medication event;
storing the medication event as part of a history of medication events; and
producing, via the IMM system, an alert when the amount of the supply of the medication is less than a predetermined level.

2. The method of claim 1, further comprising:
tracking a time period after the medication event occurs before a subsequent medication event occurs; and
producing, via the IMM system, an alert when the time period exceeds a predetermined threshold.

3. The method of claim 2, further comprising:
receiving, via the IMM system, the predetermined threshold.

4. The method of claim 1, further comprising:
sending the history of medication events to a computing device located at a healthcare professional premises.

5. The method of claim 1, further comprising:
sending a medication refill request to a computing device located at a medication provider premises when the amount of the supply of the medication is less than or equal to a second predetermined level.

6. The method of claim 5, further comprising:
after sending the medication refill request, receiving a refill notification from the computing device indicating that a replacement supply of the medication has been prepared; and
producing, via the IMM system, an alert when the refill notification is received.

7. The method of claim 1, wherein the medication is a first medication, the method further comprising:
receiving, via the IMM system, at least one new medication event associated with a second medication that is different than the first medication;
determining, based on the new medication event, interactions between the first and second medications; and
producing an output indicative of the interactions.

8. The method of claim 7, further comprising:
sending the new medication event to a computing device located at a healthcare professional premises.

10. The method of claim 1, wherein the at least one medication event is received from the patient via a cellular device.

11. The method of claim 1, further comprising:
receiving, via the IMM system, a medication inquiry from the patient;
identifying, based on the medication inquiry, a request for a medication parameter;
identifying a value corresponding to the medication parameter; and
producing an output indicative of the value.

12. A method for analyzing medical information, the method comprising:
receiving, via an interactive medical management (IMM) system, a medical parameter of a patient from a remote medical device;
storing the medical parameter as part of a medical profile of the patient; and
sending the medical profile to a computing device located at a healthcare professional premises.

13. The method of claim 12, wherein the remote medical device is a thermometer, stethoscope, oxygen sensor, pulsometer, heart monitor, breathing monitor, blood pressure monitor, blood oxygen monitor, blood sugar monitor, deep breathing lung exerciser, pulse oximeter, or breath measurement system.

14. The method of claim 12, further comprising:
simultaneously transmitting, via the IMM system, communication signals between the healthcare professional and the patient.

15. An interactive medical management (IMM) system, comprising:
a processor; and
at least one storage device storing instructions that, when executed cause the processor to perform a process, the process comprising:
receiving at least one medication event associated with a medication of a patient;
determining an amount of a supply of the medication possessed by the patient based on the medication event;
storing the medication event as part of a history of medication events; and
producing an alert when the amount of the supply of the medication is less than a predetermined level.

16. The IMM system of claim 15, wherein the alert is produced by a display or a speaker.

17. The IMM system of claim 15, wherein the process further comprises:
tracking a time period after the medication event occurs before a subsequent medication event occurs; and
producing an alert when the time period exceeds a predetermined threshold.

18. The IMM system of claim 15, wherein the process further comprises:
sending a medication refill request to a computing device located at a medication provider premises when the amount of the supply of the medication is less than or equal to a second predetermined level.

19. The IMM system of claim 15, wherein the process further comprises:
receiving a medical parameter of a patient from a remote medical device;
storing the medical parameter as part of a medical profile of the patient; and
sending the medical profile to a computing device located at a healthcare professional premises.

20. The IMM system of claim 19, wherein the remote medical device is a thermometer, stethoscope, oxygen sensor, pulsometer, heart monitor, breathing monitor, blood pressure monitor, blood oxygen monitor, blood sugar monitor, deep breathing lung exerciser, pulse oximeter, or a breath measurement system.
